(19) United States
(12) Patent Application Publication
(10) Pub. No.: US 2022/0273355 A1
(43) Pub. Date: Sep. 1, 2022

(54) MEDICAL INSTRUMENT

(71) Applicant: OLYMPUS WINTER & IBE GMBH, Hamburg (DE)

(72) Inventor: Ralf KÖNEMANN, Potsdam (DE)

(73) Assignee: OLYMPUS WINTER & IBE GMBH, Hamburg (DE)

(21) Appl. No.: 17/682,035

(22) Filed: Feb. 28, 2022

(30) Foreign Application Priority Data
Mar. 1, 2021 (DE) 10 2021 104 865.6

(51) Int. Cl. A61B 18/14

(52) U.S. Cl. CPC A61B 18/14; A61B 2018/00011

(57) ABSTRACT

A medical instrument is presented having a proximal main body and an elongated shaft disposed distally of the main body, wherein the shaft includes a first shaft tube and a second shaft tube coaxially arranged around the first shaft tube, the interior of the first shaft tube forming a first fluid space, and an annular space between the first shaft tube and the second shaft tube forming a second fluid space. The instrument wherein the proximal ends of the first shaft tube and the second shaft tube are received in a common retaining body arranged in the main body, wherein the retaining body includes a first supply channel to the first fluid space and a second supply channel to the second fluid space and is configured as a one-piece injection moulded body.

The invention relates to a medical instrument having a proximal main body and an elongated shaft disposed distally of the main body, wherein the shaft comprises a first shaft tube and a second shaft tube coaxially arranged around the first shaft tube, the interior of the first shaft tube forming a first fluid space, and an annular space between the first shaft tube and the second shaft tube forming a second fluid space.

Corresponding instruments are used in modern medicine, for example, as probes to examine and/or treat an area of interest of tissue. For example, fluids may be introduced into or withdrawn from the tissue of interest via the fluid spaces. The fluid spaces can likewise be used to pass a cooling fluid through the instrument. This can prevent overheating and premature drying of the tissue of interest, for example in electrosurgical applications.

Such probes are expensive to manufacture. This is mainly due to the fact that a reliable and tight connection of fluid lines to the respective fluid spaces requires several operations in which individual components have to be assembled one after the other and then sealed. The corresponding steps are time-consuming and susceptible to errors.

It is therefore an object of the invention to provide an improved medical instrument.

According to the invention, this object is achieved by a medical instrument having a proximal main body and an elongated shaft disposed distally of the main body, wherein the shaft comprises a first shaft tube and a second shaft tube coaxially arranged around the first shaft tube, the interior of the first shaft tube forming a first fluid space, and an annular space between the first shaft tube and the second shaft tube forming a second fluid space, which is further formed in that the proximal ends of the first shaft tube and the second shaft tube are received in a common retaining body arranged in the main body, wherein the retaining body comprises a first supply channel to the first fluid space and a second supply channel to the second fluid space and is configured as a one-piece injection moulded body.

By designing the retaining body as a one-piece injection moulded body, the number of assembly steps required may be reduced to a minimum.

In a preferred embodiment of a medical instrument according to the invention, the retaining body may comprise a stepped receiving channel for the first and second shaft tubes. For the purposes of the invention, a “stepped” receiving channel is understood to be, in particular, a receiving channel which comprises a first inner diameter in a first longitudinal section, and which comprises a second inner diameter in a second longitudinal section which is different from the first inner diameter. The transition between the first longitudinal section and the second longitudinal section may be designed as a right-angled step. Alternatively, the transition may comprise one or more chamfers and/or roundings. Regardless of the exact configuration of the transition, it will be referred to as a “step” in the context of the present invention.

In a possible further embodiment of a medical instrument according to the invention, the receiving channel may proximally merge into the first supply channel. The first supply channel may thus be configured as an extension of the receiving channel.

The inner diameter of the receiving channel proximal to the step may approximately correspond to an outer diameter of the first shaft tube. The first shaft tube may thus be received in the proximal portion of the receiving channel.

The first shaft tube may be connected to the retaining body by overmoulding, adhesive bonding, friction welding, hot embedding, or hydraulic grouting. Appropriate connection techniques can be used to achieve secure and uncomplicated sealing reception of the first shaft tube in the receiving channel.

The second supply channel may merge into the receiving channel in the area of the step. In this way, a connection between the second supply channel and the second fluid space can be easily established.

The inner diameter of the receiving channel distal from the step may be larger than an outer diameter of the second shaft tube. Furthermore, the receiving channel may comprise one or more protrusions distal from the step. Thus, the second shaft tube may be received with clearance in the distal portion of the receiving channel, and may be held in the correct position by the protrusions of the receiving channel.

In a particularly preferred embodiment of a medical instrument according to the invention, the second shaft tube may be adhesively bonded to the retaining body. In this regard, an adhesive may be introduced between the second shaft tube and the retaining channel so that the second shaft tube is sealingly fixed in the retaining channel.

In one possible embodiment of a medical instrument according to the invention, the shaft may include one or more first openings connecting the second fluid space to the exterior of the instrument. Through the first openings, a fluid may be delivered from the second fluid space into a tissue into which the medical instrument is pierced. Similarly, a medium may be aspirated from the tissue through the first openings. The first openings may be disposed along the entire length of the second shaft tube or only in a distal portion of the second shaft tube.

In another possible embodiment of a medical instrument according to the invention, the shaft may comprise one or more second openings connecting the first fluid space to the exterior of the instrument. The second openings, similar to the first openings described above, may be used to deliver and/or aspirate a fluid into and/or out of the tissue. Preferably, the second openings are located at the distal end of the first shaft tube.

In another possible embodiment of a medical instrument according to the invention, at the distal end of the shaft there may be a connection between the first fluid space and the second fluid space. A corresponding embodiment allows circulation of a fluid through the medical instrument.

According to a further embodiment of the invention, the shaft of a medical instrument may comprise a third shaft tube arranged around the second shaft tube.

In this regard, the third shaft tube may comprise an electrically conductive material, and a first electrode may be arranged on the shaft which is electrically conductively connected to the third shaft tube.

Alternatively or additionally, the second shaft tube may also comprise an electrically conductive material, and a second electrode may be arranged on the shaft, which is electrically conductively connected to the second shaft tube.

By taking the appropriate measures, the medical instrument can be easily implemented as a monopolar or bipolar electrosurgical instrument. In this regard, the second and/or the third shaft tube function as electrical leads to the electrodes, so that no separate leads need to be laid in the narrow shaft.

The invention is explained in more detail below with reference to a number of exemplary embodiments. In this connection, the illustrated embodiments serve merely to provide a better understanding of the invention without limiting it.

Showing:

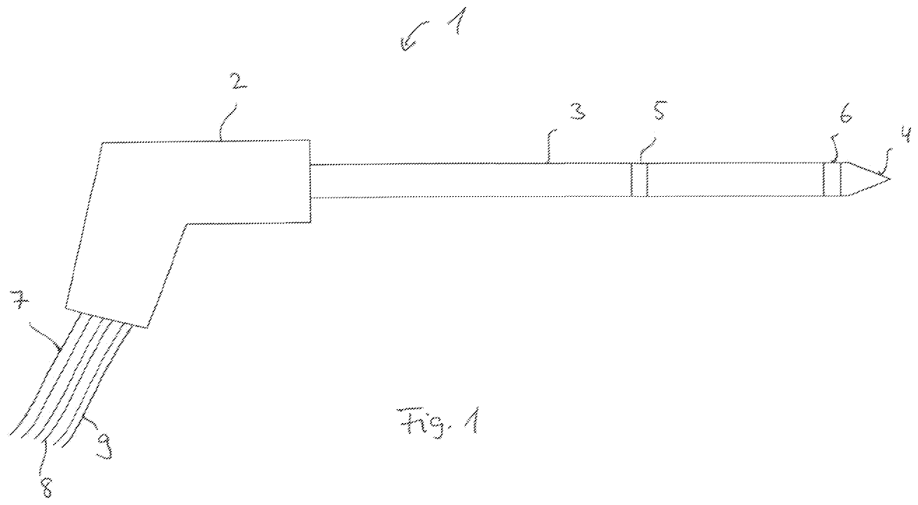

FIG. 1: a medical instrument,

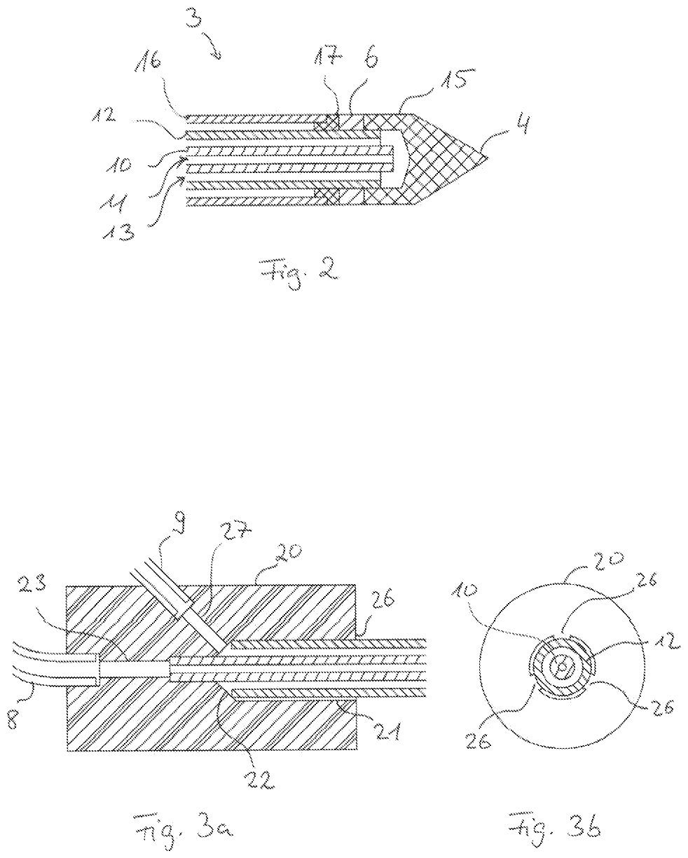

FIG. 2: a distal end portion of an instrument,

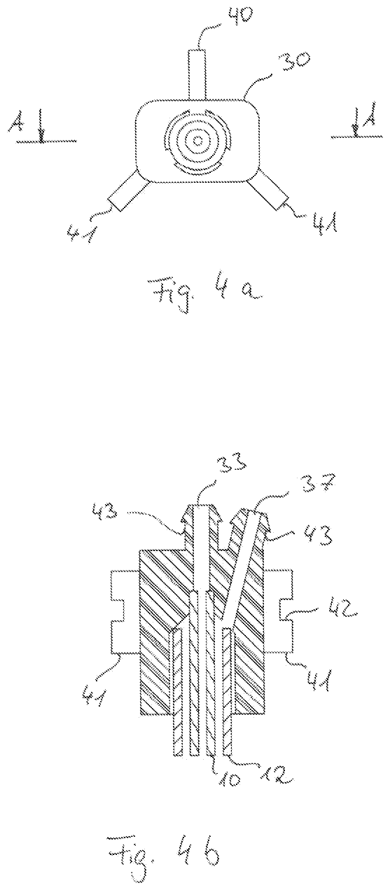

FIG. 3a and FIG. 3b: a retaining body,

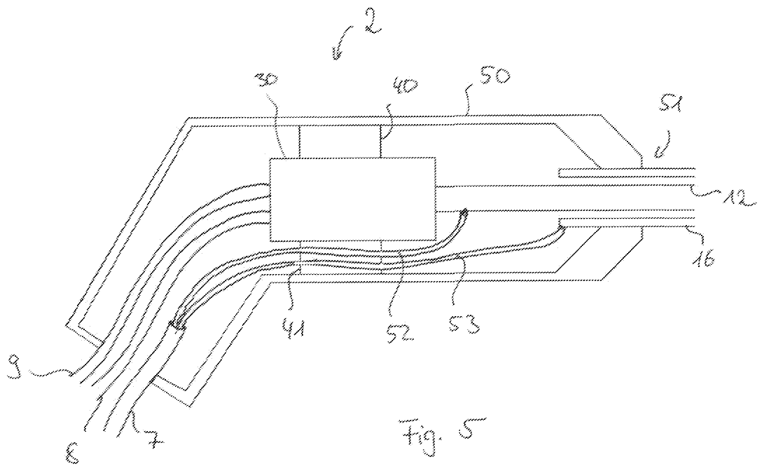

FIG. 4a and FIG. 4b: another retaining body,

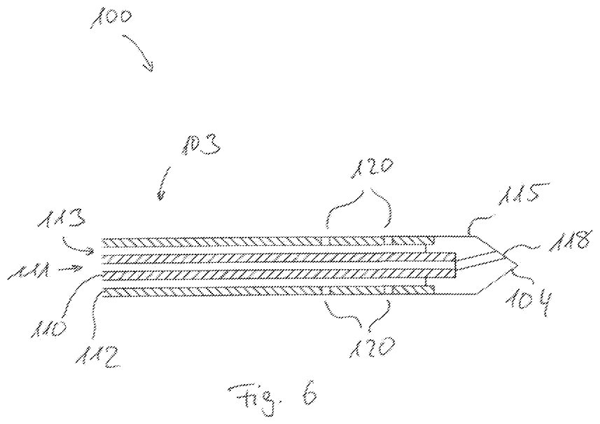

FIG. 5: a main body,

FIG. 6: another medical instrument.

In FIG. 1, a medical instrument 1 is shown. The medical instrument 1 shown is an electrosurgical insertion probe with integrated cooling.

The medical instrument 1 includes a proximal main body 2 and an elongated shaft 3 disposed distally of the main body 2.

In this context, the terms “proximal” and “distal” are to be understood as meaning that a “proximal” portion of the instrument 1 is located close to an operator of the instrument 1, for example a medical professional, while a “distal” portion of the instrument 1 is located away from the operator and thus faces the patient to be treated.

The shaft 3 has a tip 4 with which the shaft 3 can be pierced into tissue to be treated (not shown).

Electrodes 5, 6 are arranged along the shaft 3. Via the electrodes 5, 6, electrosurgical therapy signals can be delivered to a tissue into which the shaft 3 is inserted. The electrosurgical therapy signals may, for example, sclerotize or coagulate tissue.

The electrodes 5, 6 may be connected via a connecting cable 7 to an electrosurgical generator, not shown, which generates and controls electrosurgical therapy signals.

In order to avoid overheating of the tissue to be treated and the resulting premature drying out, the instrument 1 may be cooled. For this purpose, cooling liquid may be supplied and discharged via hoses 8, 9.

In FIG. 2, a distal end portion of the instrument 1 is shown in a schematic sectional view. It can be seen that the shaft 3 comprises three coaxial shaft tubes.

An interior of a first shaft tube 10 forms a first fluid space 11. A second fluid space 13 is formed between the first shaft tube and a second shaft tube 12. An end piece 15 is placed on the second shaft tube 12, forming the tip 4 of the shaft 3. The end piece 15 provides a fluidic connection between the first fluid chamber 11 and the second fluid chamber 13. The end piece 15 may be made of non-conductive material or may be provided with an insulating coating.

Proximal of the end piece 15, the electrode 6 is attached to the second shaft tube 12. The electrode 6 may, for example, be a ring of conductive material. The second shaft tube 12 is also made of conductive material and represents an electrical supply lead to the electrode 6.

The second shaft tube 12 is surrounded by a third shaft tube 16. The third shaft tube 16 is also made of conductive material, and an insulating ring 17 prevents contact between the third shaft tube 16 and the electrode 6 and the second shaft tube 12. The third shaft tube 16 may be provided with an insulating coating along its length, which is not shown. An opening in this coating may form the electrode 5, which is not shown in FIG. 2.

To cool the instrument 1 during treatment, a cooling fluid is directed into the first fluid space 11 in the main body 2 via the hose 8. The cooling fluid runs in the first fluid space 11 to the distal end of the shaft 3, where it passes through the end piece 15 into the second fluid space 13. In the second fluid space 13, the cooling fluid then runs back to the main body 2, from where it is discharged through the hose 9.

The cooling fluid is supplied to and discharged from the fluid spaces in the main body 2 via a retaining body. A possible design of the retaining body is shown in FIGS. 3a, 3b.

FIG. 3a shows a retaining body 20 in longitudinal section. The retaining body 20 is designed as a one-piece injection moulded part and has a stepped receiving bore 21 in which the first and second shaft tubes 10, 12 are received. At a step 22 of the receiving bore 21, which is disposed between a proximal portion and a distal portion of the receiving bore 21, an inner diameter of the receiving bore 21 changes.

Proximally of the step 22, the inner diameter of the receiving bore 21 is approximately equal to an outer diameter of the first shaft tube 10, whereas the first shaft tube 10 is sealingly received in the receiving bore 21.

The first shaft tube 10 is preferably connected to the retaining body 20 by overmolding. For this purpose, the first shaft tube 10 is inserted into an injection mould in such a way that its proximal end is enclosed by the material of the retaining body 20 during the injection moulding process. Alternatively, the first shaft tube 10 can be inserted into the receiving bore after the retaining body 20 has been produced and fixed there by suitable joining processes. Suitable joining methods include adhesive bonding, friction welding, and hydraulic grouting, to name a few.

At its proximal end, the receiving bore 21 merges into a first supply channel 23. The first supply channel 23 connects the first fluid space 11 inside the first shaft tube 10 to an end face 25 of the retaining body 20. Here, the first supply channel 23 has a flare into which a distal end of the supply hose 8 can be inserted. For attachment purposes, the supply tube 8 may have elastic projections not shown.

Distal from the step 22, the inner diameter of the receiving bore 21 is slightly larger than an outer diameter of the second shaft tube 12, and has projections 26 in the longitudinal axial direction. The projections 26 are arranged to center the second shaft tube 12 in the receiving bore 23. Remaining segmental gaps between the second shaft tube 12 and the receiving bore 23 are filled with adhesive and/or provided with a seal for fixing and sealing.

In the area of the step 22, a second supply channel 27 merges into the receiving bore 23. The second supply channel is thus in communication with the second fluid space 13. Distally, the second supply bore 27 ends at an outer side of the retaining body 20, where it has a widening similar to the supply bore 23 for receiving the discharge hose 9.

FIG. 3b shows the retaining body 20 from a distal direction. Here the contour of the projections 26 is visible.

The described design of the retaining body 20 enables simple and secure assembly of the shaft tubes 10, 12 and the hoses 8, 9, while ensuring reliable sealing against leakage from the hose connections and between the fluid spaces.

FIGS. 4a, 4b show another embodiment of a retaining body 30. Here, FIG. 4a shows a front view from a distal direction, while FIG. 4b shows a sectional view from the direction of arrows “A” in FIG. 4a.

The retaining body 30 differs from the previously described retaining body 20 essentially only in its external shape. For the sake of clarity, elements having the same effect are therefore each given a reference symbol raised by 10 and are not described again in detail.

The retaining body 30 has wing-like positioning elements 40, 41. The positioning elements 40, 41 serve to support the retaining body 30 in the main body 2 of the instrument 1. In this respect, they are arranged in such a way that the entire retaining body 30 may be produced by injection molding using a two-part injection mold. The positioning elements 41 also have a recess 42, which allow the retaining body 30 to be aligned in the longitudinal axial direction.

The supply channels 33, 37 of the retaining body 30 end in push-on stubs 43, onto which the hoses 8, 9 not shown in FIG. 4b can be pushed.

The shaft tubes 10, 12 are attached and sealed in the same way as described above for the retaining body 20.

In FIG. 5, the structure of the main body 2 of the instrument 1 is shown schematically. The main body 2 comprises a housing 50, in which the retaining body 30 is arranged. The retaining body 30 is supported by the positioning elements 40, 41 on the housing.

The distal end of the housing 50 has an opening in which the third shaft tube 16 is firmly received, for example by adhesive bonding. The second shaft tube 12 as well as the first shaft tube, which is not visible in FIG. 5, protrude into the third shaft tube 16.

Leads 52, 53 of the connecting cable 7 are connected to the second shaft tube 12 and to the third shaft tube 16, respectively. Contacting of the shaft tubes 12, 16 with the leads 52, 53 may be made by known means, for example by soldering, clamping, crimping, or the like.

The hoses 8, 9 are connected to the retaining body 30, and the hose connection stubs 43 are not shown in detail.

After connecting the hoses 8, 9 and the leads 52, 53, the housing 50 of the main body 2 is filled with potting compound. This relieves the respective connection points against tension, electrically insulates them, and protects them against moisture.

In FIG. 6, another medical instrument 100 is partially shown. Unlike the instrument 1, the shaft 103 of the instrument 100 comprises only a first shaft tube 110 with a first fluid space 111 enclosed thereby, and a second shaft tube 112 enclosing a second fluid space 113 around the first shaft tube 110.

The shaft 103 is again capped by an end piece 115, which forms a tip 104. Unlike the end piece 15 of the instrument 1, the end piece 115 seals the first fluid space 111 and the second fluid space 113 from each other.

A channel 118 in the end piece 115 connects the first fluid space 111 to the exterior of the instrument 100.

The second shaft tube 112 of the instrument 100 includes openings 120 through which the second fluid space 113 communicates with the exterior of the instrument 100.

Through the channel 118 and the openings 120, it is possible to introduce and/or remove media from a tissue to be treated. For example, fluid samples may be removed selectively through the channel 118 or, in a distributed manner, through the openings 120. Similarly, therapeutics or other media may be injected into the tissue in a targeted or distributed manner. It is also possible to combine the removal and delivery of media.

1. A medical instrument having a proximal main body and an elongated shaft disposed distally of the main body, wherein
the shaft comprises a first shaft tube and a second shaft tube coaxially arranged around the first shaft tube,
the interior of the first shaft tube forming a first fluid space, and
an annular space between the first shaft tube and the second shaft tube forming a second fluid space,
wherein
the proximal ends of the first shaft tube and the second shaft tube are received in a common retaining body arranged in the main body, wherein the retaining body comprises a first supply channel to the first fluid space and a second supply channel to the second fluid space and is configured as a one-piece injection moulded body.

2. The medical instrument according to claim 1, wherein the retaining body comprises a stepped receiving channel for the first and the second shaft tubes.

3. The medical instrument according to claim 2, wherein the receiving channel proximally merges into the first supply channel.

4. The medical instrument according to claim 2, wherein an inner diameter of the receiving channel proximal to a step approximately corresponds to an outer diameter of the first shaft tube.

5. The medical instrument according to claim 4, wherein the first shaft tube is connected to the retaining body by overmoulding, adhesive bonding, friction welding, hot embedding, or hydraulic grouting.

6. The medical instrument according to claim 2, wherein the second supply channel merges into the receiving channel in the area of the step.

7. The medical instrument according to claim 2, wherein an inner diameter of the receiving channel distal from the step is larger than an outer diameter of the second shaft tube.

8. The medical instrument of claim 7, wherein the receiving channel comprises one or more protrusions distal from the step.

9. The medical instrument according to claim 7 wherein the second shaft tube is adhesively bonded to the retaining body.

10. The medical instrument according to claim 1, wherein the shaft comprises one or more first openings connecting the second fluid space to the exterior of the instrument.

11. The medical instrument according to claim 1, wherein the shaft comprises one or more second openings connecting the first fluid space to the exterior of the instrument.

12. The medical instrument according to claim 1, wherein at the distal end of the shaft there is a connection between the first fluid space and the second fluid space.

13. The medical instrument according to claim 1, wherein the shaft comprises a third shaft tube arranged around the second shaft tube.

14. The medical instrument according to claim 13, wherein the third shaft tube comprises an electrically conductive material, and in that a first electrode is arranged on the shaft, which is electrically conductively connected to the third shaft tube.

15. The medical instrument according to claim 1, wherein the second shaft tube comprises an electrically conductive material, and in that a second electrode is arranged on the shaft, which is electrically conductively connected to the second shaft tube.
